(19) United States
(12) Patent Application Publication
(10) Pub. No.: US 2015/0164314 A1
(43) Pub. Date: Jun. 18, 2015

(54) STEERABLE LASER PROBE

(71) Applicant: Medical Instrument Development Laboratories, Inc., San Leandro, CA (US)

(72) Inventor: Erik William Peterson, Walnut Creek, CA (US)

(73) Assignee: Medical Instrument Development Laboratories, Inc., San Leandro, CA (US)

(21) Appl. No.: 14/107,803

(22) Filed: Dec. 16, 2013

(51) Int. Cl. A61B 3/00

(52) U.S. Cl. CPC A61B 3/0008

(57) ABSTRACT

A steerable laser probe including an optical fiber, a flexible tubular sleeve positioned co-axially with the flexible tubular sleeve relative to an axis, and a rigid tubular sleeve positioned co-axially with the flexible tubular sleeve and the optical fiber relative to the axis, and positioned between the flexible tubular sleeve and the optical fiber along the axis.

### BACKGROUND

The present invention relates to laser probes used in ophthalmologic surgeries. More particularly, embodiments of the invention are related to laser probes which are capable of bending to send light into areas typically not accessible with straight laser probes.

### SUMMARY

In one embodiment, the invention provides a steerable laser probe including a flexible tubular sleeve, an optical fiber positioned co-axially with the flexible tubular sleeve relative to an axis, and a rigid tubular sleeve positioned co-axially with the flexible tubular sleeve and the optical fiber relative to the axis. The rigid tubular sleeve is positioned between the flexible tubular sleeve and the optical fiber along the axis. The rigid tubular sleeve is movable along the axis relative to the flexible tubular sleeve and the optical fiber, between a first position and a second position. When the rigid tubular sleeve is in the first position, the rigid tubular sleeve maintains a portion of the flexible tubular sleeve in a substantially straight position and when the rigid tubular sleeve is in the second position, the absence of the rigid tubular sleeve allows the portion of the flexible tubular sleeve to curve.

Other aspects of the invention will become apparent by consideration of the detailed description and accompanying drawings.

### BRIEF DESCRIPTION OF THE DRAWINGS

FIG. 1 is a cross-sectional view of a steerable laser probe according to one embodiment of the invention.

FIG. 2 shows the steerable laser probe of FIG. 1 in a first position.

FIG. 3 shows the steerable laser probe of FIG. 1 in a second position.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

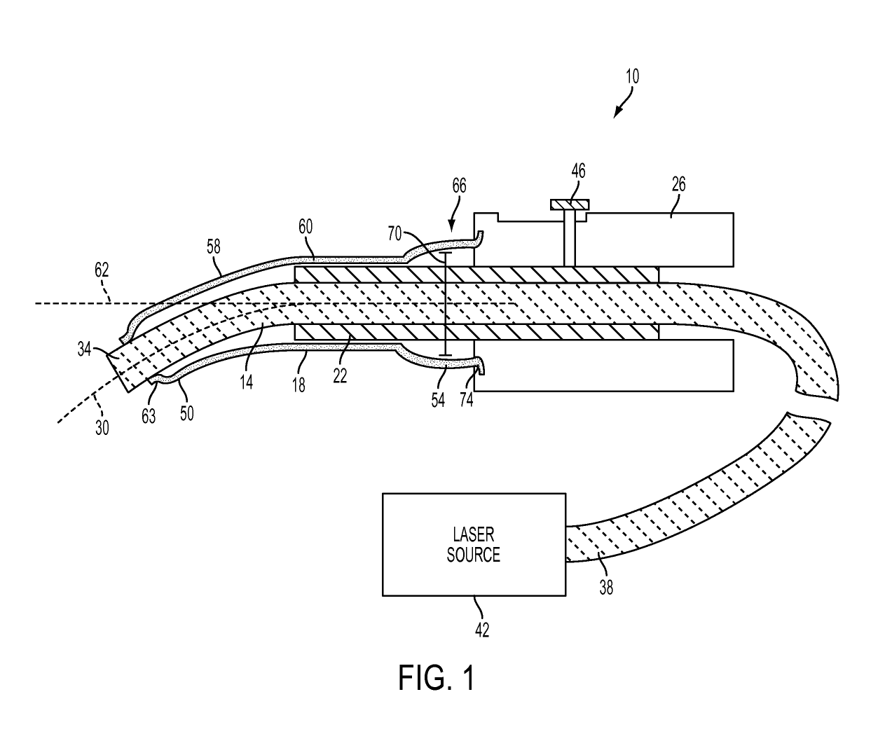

FIG. 1 illustrates one embodiment or example of a steerable laser probe 10. In the embodiment shown, the steerable laser probe 10 includes an optical fiber 14, a flexible tubular sleeve 18, a rigid tubular sleeve 22, and a handle 26. The flexible tubular sleeve 18, the optical fiber 14, and the rigid tubular sleeve 22 are positioned co-axially with respect to one another and to an axis 30. The optical fiber 14 includes a proximal end 34 and a distal end 38. The distal end 38 of the optical fiber 14 extends beyond the handle 26 and is connected to a laser source 42. The proximal end 34 of the optical fiber 14 is used to direct laser energy to a specific area. In some embodiments, the optical fiber 14 is used to illuminate parts of the human body that are inaccessible to external light sources.

The optical fiber 14 is positioned within the rigid tubular sleeve 22, and the rigid tubular sleeve 22 is positioned within the flexible tubular sleeve 18. The handle 26 is coupled with the flexible tubular sleeve 18 such that the handle is able to control the depth of insertion of the steerable laser probe. The rigid tubular sleeve 22 is movable within the handle 26 and the flexible tubular sleeve 18. The handle 26 includes an actuator 46 that controls the movement of the rigid tubular sleeve 22. The actuator 46 is connected to the rigid tubular sleeve 22 and is configured to move the rigid tubular sleeve 22 relative to the flexible tubular sleeve 18 and the optical fiber 14. The flexible tubular sleeve 18 interacts with the optical fiber 14, such that the actuator 46 is able to control the direction of the laser energy from the optical fiber 14.

The optical fiber 14 guides energy (in the form of light) from the laser source 42. Thus, the location and orientation or positioning of the optical fiber determines the particular location to which light from the laser source is directed. Bending or curving of the optical fiber 14 changes the direction of the light and, ultimately, the location to which the light is directed. The flexible tubular sleeve 18 includes a proximal end 50 and a distal end 54. The flexible tubular sleeve 18 includes a curved portion 58 which extends from a portion 60 to the proximal end 50. In the illustrated embodiment, the flexible tubular sleeve 18 also includes a taper 63 near the proximal end 50 of the flexible tubular sleeve 18. The taper 63 contacts and holds the optical fiber 14 so that longitudinal movement of the optical fiber 14 with respect to the flexible tubular sleeve 18 is inhibited. In addition, since both the optical fiber 14 and flexible tubular sleeve 18 are flexible, straightening of the flexible tubular sleeve 18 causes a corresponding straightening of the optical fiber 14. Further, when the sleeve 18 is not straightened (or in its natural or default state), the optical fiber is curved, matching the curve of the sleeve 18. When the optical fiber 14 is so curved, light from the laser source may be directed at an angle from the horizontal axis 62.

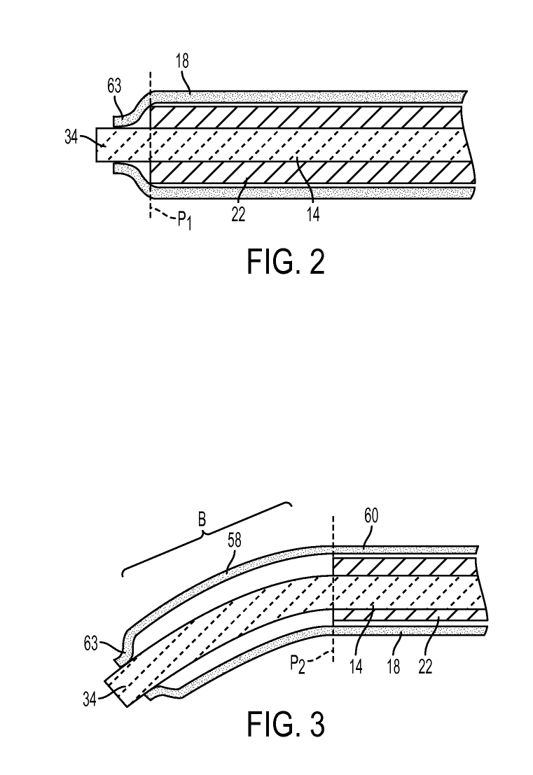

The rigid tubular sleeve 22 is positioned between the optical fiber 14 and the flexible tubular sleeve 18. In the embodiment shown, the rigid tubular sleeve 22 is substantially straight. The rigid tubular sleeve 22 is movable, via the actuator 46, between a first position (P1) (FIG. 2) and a second position (P2) (FIG. 3). As shown in FIG. 2, in the first position P1 the rigid tubular sleeve 22 is extended towards the proximal end 50 of the flexible tubular sleeve 18. In the first position P1, the rigid tubular sleeve 22 inhibits the flexible tubular sleeve 18 from curving due to the substantially straight shape and rigid characteristic of the rigid tubular sleeve 22. Said in another way, the rigid tubular tube 22 straightens the sleeve 18.

As shown in FIG. 3, in the second position P2, the rigid tubular sleeve 22 retracts or moves toward the handle 26 and the absence of the rigid tubular sleeve 22 in the curved portion 58 of the flexible tubular sleeve 18 allows the flexible tubular sleeve 18 to curve according to its predetermined shape or natural tendencies. A user inserts the steerable laser probe 10 into an area, such as a cavity in the eye, while the rigid tubular sleeve 22 is in the first position. The user changes the angle of the projected laser energy from the optical fiber 14 by retracting the rigid tubular sleeve 22 to the second position. When the steerable laser probe 10 needs to be removed from the area, the rigid tubular sleeve 22 is moved back to the first position to inhibit the optical fiber 14 from curving, and the steerable laser probe 10 is then easily removed from the area.

In the illustrated embodiment, the curved portion 58 of the flexible tubular sleeve 18 has a particular bend B. However, it should be understood that the flexible tubular sleeve 18 may be bent in other ways and shapes.

In the illustrated embodiment, the flexible tubular sleeve 18 includes an attachment feature 66 near the distal end 54 of the flexible tubular sleeve 18 that allows the flexible tubular sleeve 18 to be attached to the handle 26. The attachment feature 66 includes an expanded diameter 70 and a snap feature 74. The expanded diameter 70 allows the flexible tubular sleeve 18 to be coupled to the handle 26, and the snap feature 74 secures the flexible tubular sleeve 18 to the handle 26.

In prior-art devices, steerable laser probes often position a flexible tube inside a rigid tube. The flexible tube moves from a retracted position to an extended position. In the retracted position, the flexible tube is co-axially positioned inside the rigid tube and inhibited from curving. However, in the extended position, the flexible tube moves past the rigid tube and is able to bend. However, as the flexible tube bends or curves, the flexible tube also experiences longitudinal displacement. A user has to account for the longitudinal displacement to direct the light from an optical fiber in a desired direction and such steerable probes require the user to adjust the depth of insertion of the probe.

Since the steerable laser probe 10 includes the rigid tubular sleeve 22 in between the optical fiber 14 and the flexible tubular sleeve 18, and the rigid tubular sleeve 22 retracts relative to the optical fiber 14 and the flexible tubular sleeve 18, the flexible tubular sleeve 18 bends or curves without experiencing longitudinal displacement. As a consequence, the steerable laser probe 10 provides a user with an easy way to direct laser energy from the optical fiber 14 to a desired location without requiring adjustment of the depth of insertion of the steerable laser probe 10 due to longitudinal displacement.

In some embodiments, the flexible tubular sleeve 18 is made from a polyimide resin, such as Kapton. Kapton is used to make tubes with extremely thin walls. Generally uncured resin is coated onto a particular form by dipping the form into the liquid resin. The form defines the shape of the curve of the flexible tubular sleeve. The layer of resin is then cured. In some embodiments, the resin is cured by heating. Typically, multiple layers are built up by dipping the form in the resin and curing the layer of resin. Finally, when sufficient layers have been cured, the form is removed. In some embodiments, the form is made from a soluble material to facilitate removal of the form. The form for the flexible tubular sleeve 18 includes a curved portion that defines the curved portion 58 of the flexible tubular sleeve 18. In some embodiments, the form also includes a tapered end to form the tapered end 63 and/or an expanded diameter to form the attachment feature 66.

In other embodiments, the flexible tubular sleeve is made from a thermo-form polymer, for example, polyetheretherketone (PEEK). As described above for polymide resin, the form for the flexible tubular sleeve 18 includes a curved portion that defines the curved portion 58 of the flexible tubular sleeve 18. In some embodiments, the form made of thermo-form polymer also includes a reduction in diameter at an end of the form that defines the tapered end 63 and/or an expanded diameter to form the attachment feature 66. The flexible tubular sleeve 18 can also be made from other flexible materials.

Thus, the invention provides, among other things, a steerable laser probe that inhibits longitudinal displacement of the optical fiber while changing the angle at which light is directed. Various features and advantages of the invention are set forth in the following claims.

What is claimed is:

1. A steerable laser probe comprising:
an optical fiber;
a flexible tubular sleeve positioned co-axially with the optical fiber relative to an axis; and
a rigid tubular sleeve positioned co-axially with the flexible tubular sleeve and the optical fiber relative to the axis, and positioned between the flexible tubular sleeve and the optical fiber along the axis.

2. The steerable laser probe of claim 1, wherein the rigid tubular sleeve is movable along the axis relative to the flexible tubular sleeve and the optical fiber between a first position and a second position, wherein, when the rigid tubular sleeve is in the first position, the rigid tubular sleeve maintains a portion of the flexible tubular sleeve in a substantially straight position and, when the rigid tubular sleeve is in a second position, the absence of the rigid tubular sleeve allows a portion of the flexible sleeve to curve.

3. The steerable laser probe of claim 1, wherein the flexible tubular sleeve and the optical fiber are attached, such that the flexible tubular sleeve and the optical fiber do not move relative to each other.

4. The steerable laser probe of claim 1, wherein the flexible tubular sleeve includes a pre-formed curve, and wherein the optical fiber curves according to the pre-formed curve of the flexible tubular sleeve.

5. The steerable laser probe of claim 2, further comprising a handle having a mechanism that activates movement of the rigid tubular sleeve between the first position and the second position.

6. The steerable laser probe of claim 1, wherein the optical fiber is connected to a light source and used to illuminate an area.

7. The steerable laser probe of claim 5, wherein the flexible tubular sleeve includes a proximal end and a distal end, and wherein the distal end of the flexible tubular sleeve is attached to the handle.

8. The steerable laser probe of claim 7, wherein the distal end of the flexible tubular sleeve includes an attachment feature to facilitate attachment of the flexible tubular sleeve to the handle.

9. The steerable laser probe of claim 7, wherein the proximal end of the flexible tubular sleeve includes a reduction in diameter to inhibit movement of the optical fiber relative to the flexible tubular sleeve.
